(19) United States
(12) Patent Application Publication
(10) Pub. No.: US 2020/0306142 A1
(43) Pub. Date: Oct. 1, 2020

(54) LABELLED ROOT CANAL FILLING MATERIAL

(71) Applicant: THE CATHOLIC UNIVERSITY OF KOREA INDUSTRY-ACADEMIC COOPERATION FOUNDATION, Seoul (KR)

(72) Inventors: Sung Seo MO, Suwon-si, Gyeonggi-do (KR); Sang Hwa LEE, Seoul (KR)

(21) Appl. No.: 16/759,205

(22) PCT Filed: Jul. 2, 2018
(86) PCT No.: PCT/KR2018/007463

§ 371 Date: Apr. 24, 2020

(30) Foreign Application Priority Data
Oct. 26, 2017 (KR) 10-2017-0139891

(51) Int. Cl. A61K 6/54; A61C 5/50; A61K 6/80; A61K 6/15

(52) U.S. Cl. CPC A61K 6/54; A61K 6/15; A61K 6/80; A61C 5/50

(57) ABSTRACT

The present disclosure relates to a labelled root canal filling material. The labelled root canal filling material according to the present disclosure is filled in a root canal to perform root canal treatment, and includes a main body having a shape corresponding to a shape of the root canal to be filled in the root canal and to be removed by a cutting tip after being inserted into the root canal; and a remaining portion integrally formed with the main body, having a mark to be distinguished from the main body, and remaining in the root canal when the main body having inserted into the root canal is removed by the cutting tip.

### TECHNICAL FIELD

The present disclosure relates to a labelled root canal filling material, and more particularly, to a labelled root canal filling material having an improved structure that may allow to check whether a filling material used for root canal treatment is filled up without voids and then enable complete endodontic treatment.

### BACKGROUND ART

A tooth which is the hardest tissue in human body has a very thin canal called a nerve canal (root canal) thereinside unlike its appearance, and nerves, blood vessels, and connective tissue called a dental pulp are contained in the nerve canal. The nerve canal in which the dental pulp is contained extends to the tip of the root of the tooth (dental root), and the dental pulp contained in the nerve canal is connected to blood vessels and nerves in a jaw bone (alveolar bone) surrounding the root of the tooth through a small opening (dental apical foramen) at the apex of the root of the tooth to enter the heart and the brain.

In general, “endodontic treatment” practically refers to “root canal treatment”. The endodontic treatment is not a process of treating and restoring damaged or infected nerves, but is a process of eliminating a dental pulp infected for various reasons, disinfecting the inside of a nerve canal (root canal), and permanently filling an empty space with another synthetic material, to cure disease and prevent any further infection.

That is, general root canal treatment involves, (a) in a state where a dental pulp is removed from a root canal A to perform treatment, (b) inserting a gutta-percha point 100 into the root canal A, (c) inserting a cutting tip 130 into the root canal A, and (d) removing a remaining portion 110, other than a portion 120 having a desired length, of the gutta-percha point 100 from the root canal A by heating and pressing the cutting tip 130, and filling (not shown) an empty space with a liquid filling and solidifying the liquid filling.

However, such a root canal treatment method has problems in that, as shown in an enlarged view of FIG. 1, although a dentist carefully and cautiously uses the cutting tip 130, according to a procedure environment, a tooth condition, or the dentist's skill, an end of the gutta-percha point 100 may fail to completely fill a root portion Al of the root canal A and may be slightly lifted from the root portion Al to have voids, and thus even after treatment, foreign materials may be introduced into the voids and retreatment may be required.

### DESCRIPTION OF EMBODIMENTS

### Technical Problem

To solve the above problems, an objective of the present disclosure is to provide a labelled root canal filling material that may allow to check whether a filling material used for root canal treatment is filled up without voids in a root portion of a root canal.

Another objective of the present disclosure is to provide a labelled root canal filling material that may allow a filling material used for root canal treatment to be filled without voids in a root portion of a root canal.

### Solution to Problem

A labelled root canal filling material according to the present disclosure for fulfilling the objectives is filled in a root canal to perform root canal treatment, and includes a main body having a shape corresponding to a shape of the root canal to be filled in the root canal and to be removed by a cutting tip after being inserted into the root canal; and a remaining portion integrally formed with the main body, having a mark to be distinguished from the main body, and remaining in the root canal when the main body having inserted into the root canal is removed by the cutting tip.

The mark of the remaining portion may be indicated by a color distinguished from a color of the main body.

The mark of the remaining portion may be indicated by a cut groove formed in a boundary portion between the main body and the remaining portion.

The remaining portion may have a cone shape whose outer diameter gradually increases from a point of a front end to a portion connected to the main body.

### Advantageous Effects of Disclosure

A labelled root canal filling material according to the present disclosure having the above configuration has advantages in that, although an incomplete filling state of a root canal occurs in a procedure, the incomplete filling state may be identified, and thus whether endodontic treatment is incomplete may be checked and re-operation or aftereffects due to wrong operation may be prevented, and an error of an initial procedure may be corrected and proper root canal treatment may be performed.

### BRIEF DESCRIPTION OF DRAWINGS

FIG. 1 is a view for describing a general root canal treatment process.

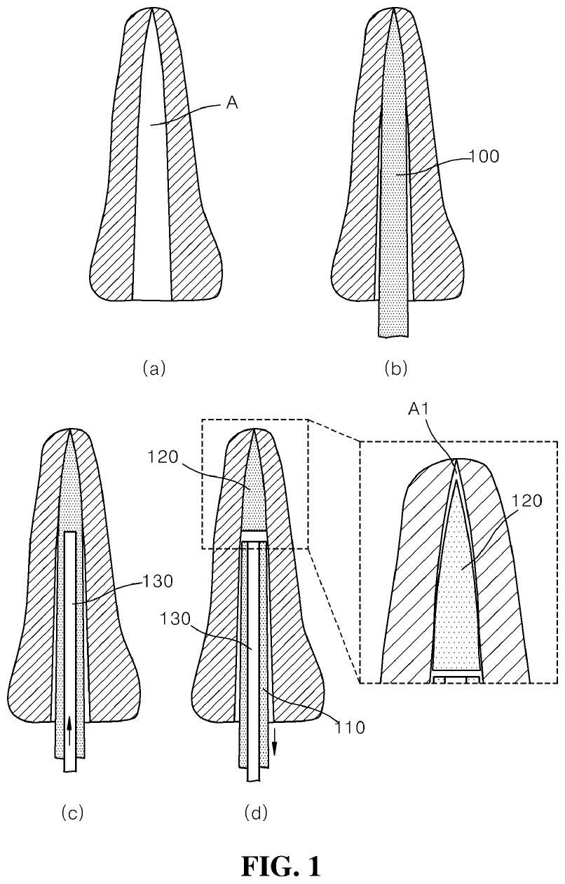

FIG. 2 is a side view of a labelled root canal filling material according to an embodiment of the present disclosure.

FIG. 3 is a view for describing a root canal treatment method using an embodiment of the present disclosure.

FIG. 4 is an enlarged view illustrating a portion IV of FIG. 3.

FIG. 5 is a view for describing advantages of an embodiment of the present disclosure.

FIG. 6 is a side view of a labelled root canal filling material according to other embodiment of the present disclosure

FIG. 7 is a view for describing advantages of other embodiment of the present disclosure.

### BEST MODE

Hereinafter, a labelled root canal filling material according to an embodiment of the present disclosure will be described in detail with the attached drawings.

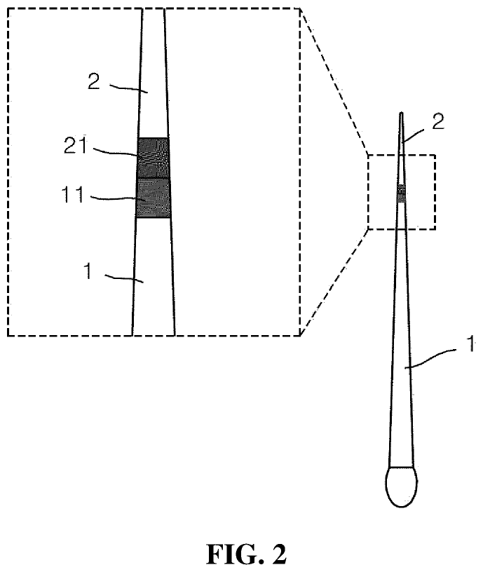

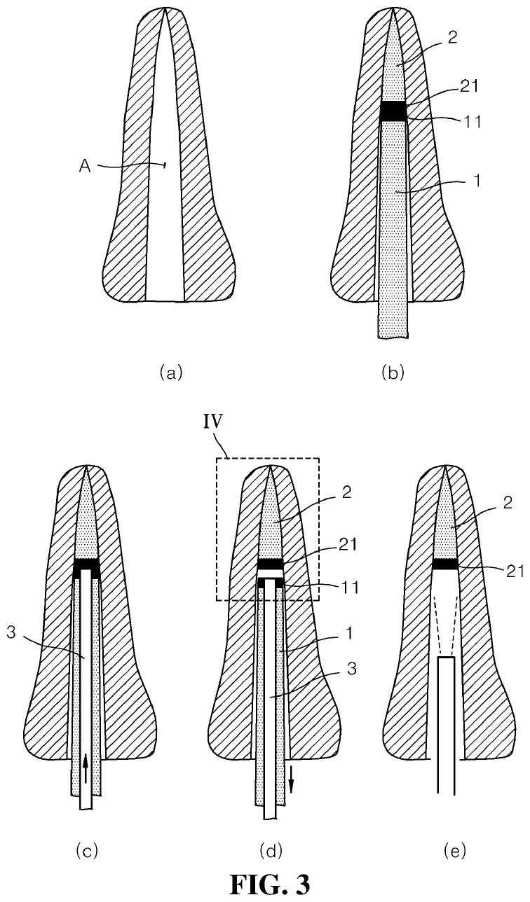

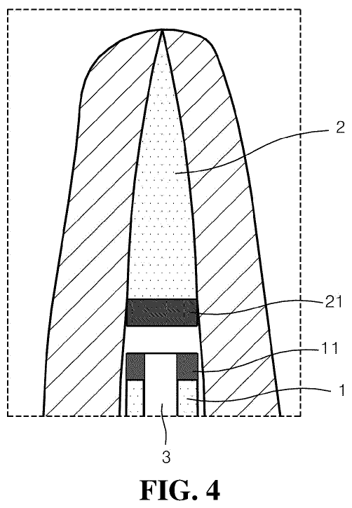

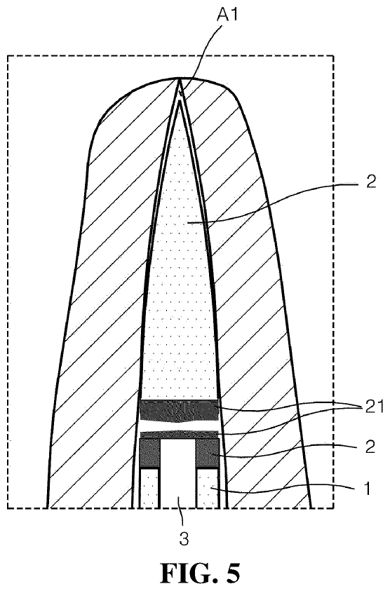

FIG. 2 is a side view of a labelled root canal filling material according to an embodiment of the present disclosure. FIG. 3 is a view for describing a root canal treatment method using an embodiment of the present disclosure. FIG. 4 is an enlarged view illustrating a portion IV of FIG. 3. FIG. 5 is a view for describing advantages of an embodiment of the present disclosure.

As shown in FIGS. 2 through 5, a labelled root canal filling material according to an embodiment of the present disclosure that is filled in a root canal A from which a dental pulp is removed for root canal treatment includes a main body 1 removed after being filled in the root canal A and a remaining portion 2 remaining in the root canal A unlike the main body 1.

The main body 1 that is to be removed by a cutting tip 3 after being inserted into the root canal A has a shape corresponding to a shape of the root canal A so that the main body is filled in the root canal A. The main body 1 may include a ready-made structure such as a gutta-percha point, or may be custom-made by a three-dimensional (3D) printer to match a shape of the root canal A that varies per person.

The gutta-percha point that is a dental material having a tapering shape and using gutta-percha as a main material is mainly used for root canal treatment (endodontic treatment), and the cutting tip 3 that is sharpened when being heated and becoming hot is a medical instrument used to cut the gutta-percha point.

The remaining portion 2 that remains in the root canal A when the main body 1 is removed by the cutting tip 3 after being inserted into the root canal A is integrally formed with the main body 1, and has a mark 21 to be distinguished from the main body 1.

A root canal treatment method using the present embodiment having the above configuration is as follows.

As well shown in FIG. 3, the root canal treatment method involves, (a) in a state where a dental pulp is removed from the root canal A to perform endodontic treatment, (b) inserting a labelled root canal filing material according to an embodiment of the present disclosure into the root canal A, (c) inserting the cutting tip 3 into the root canal A, (d) heating and pressing the cutting tip 3 to simultaneously cut the main body 1 of the present embodiment from the remaining portion 2 and remove the main body 1 from the root canal A, and (e) filling and solidifying a liquid filling in an empty space.

During such a filling process, a cutting operation as shown in (d) of FIG. 3 has to be performed by a highly skilled person precisely and carefully. This is because the remaining portion 2 remaining after the cutting operation as shown in FIG. 4 has to be filled without voids in a root portion Al of the root canal A.

When the root portion Al of the root canal A is not completely filled with the remaining portion 2 to form voids and the endodontic treatment is finished as shown in FIG. 5, a foreign material such as food waste is introduced into the voids later, thereby resulting in many aftereffects. However, according to the present embodiment, such problems may be fundamentally solved.

That is, as shown in FIG. 4, when the root portion Al of the root canal A is completely filled with the remaining portion 2 without voids, the mark 21 of the remaining portion 2 does not exist on the main body 1. In contrast, as shown in FIG. 5, a case where the root portion Al of the root canal A is not completely filled occurs when, in a process of cutting and hauling the main body 1 by using the cutting tip 3, a part of the remaining portion 2 is hauled along with the main body 1 and is cut along with the main body 1. Such a wrong cutting result may be identified by using the mark 21 existing on the main body 1.

Accordingly, according to the present embodiment, even when an incomplete filling state of the root canal A occurs in a procedure as shown in FIG. 5, the incomplete filling state may be identified by using the mark 21 existing on the main body 1, and thus it may be checked whether endodontic treatment is incomplete. Also, by pressing the remaining portion 2 to the root of the root canal A again, an error of an initial procedure may be corrected and proper root canal treatment may be performed.

The mark 21 of the remaining portion 2 used in the present embodiment is indicated by a color distinguished from the main body 1. It is preferable that the main body 1 includes a sub-mark 11 that extends from a portion (a boundary portion with the remaining portion 2) to be cut by the cutting tip 3 to a certain area and is indicated by a color different from that of the remaining portion 2.

Because the remaining portion 2 has a cone shape whose outer diameter gradually increases from a point of a front end to a portion connected to the main body 1, similarly to a structure of a gutta-percha point, it is preferable that the remaining portion 2 has a structure to be smoothly filled in the root portion Al of the root canal A.

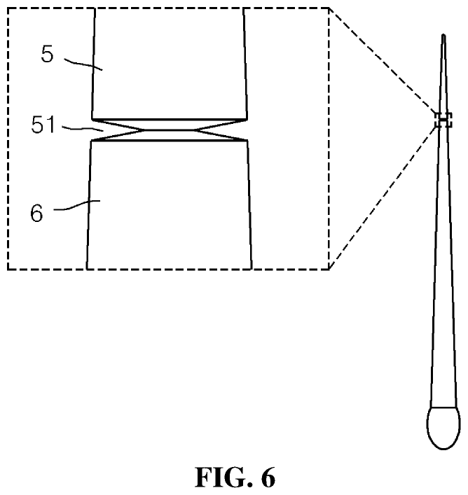

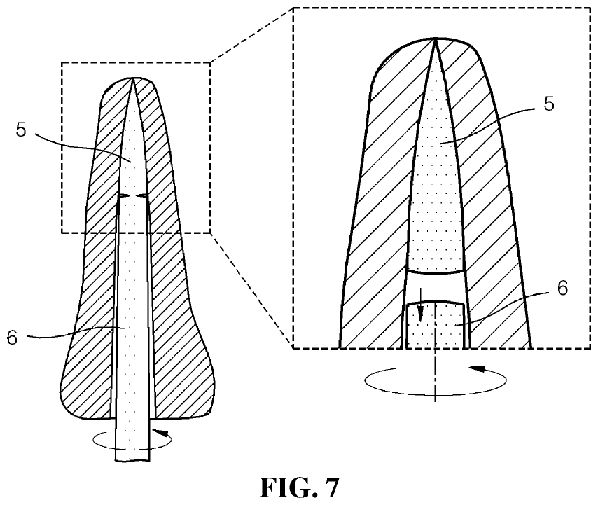

A labelled root canal filling material according to another embodiment of the present disclosure will now be described with reference to FIGS. 6 and 7.

FIG. 6 is a side view of another embodiment of the present disclosure. FIG. 7 is a view for describing a use of another embodiment of the present disclosure.

As shown in FIGS. 6 and 7, according to the preset embodiment, unlike in the above embodiment, a mark 51 of a remaining portion is indicated by a cut groove shape formed in a boundary portion between a main body 6 and a remaining portion 5.

According to the present embodiment having the configuration, as shown in FIG. 7, because, after the present embodiment is inserted into a root canal, the main body 6 is cut from the remaining portion 5 by rotating the main body 6 relative to the remaining portion 5 and the remaining portion 5 remains in the root canal while filling the root canal without voids, a cutting process using a cutting tip is not required, and thus operator convenience may be improved and the precision of root canal treatment may be improved.

While various embodiments of the present disclosure have been described, the present embodiments and the drawings attached to the specification are merely provided to explain part of the technical spirit of the present disclosure and it will be understood that all modifications or alternatives easily derived by one of ordinary skill in the art from the scope of the specification and the drawings fall within the scope of the present disclosure.

1. A labelled root canal filling material filled in a root canal for root canal treatment, the labelled root canal filling material comprising:
a main body having a shape corresponding to a shape of the root canal to be filled in the root canal, and to be removed by a cutting tip after being inserted into the root canal; and
a remaining portion integrally formed with the main body, having a mark to be distinguished from the main body, and remaining in the root canal when the main body having inserted into the root canal is removed by the cutting tip.

2. The labelled root canal filling material of claim 1, wherein the mark of the remaining portion is indicated by a color to be distinguished from a color of the main body.

3. The labelled root canal filling material of claim 1, wherein the mark of the remaining portion is indicated by a cut groove formed in a boundary portion between the main body and the remaining portion.

4. The labelled root canal filling material of claim 1, wherein the remaining portion has a cone shape whose outer diameter gradually increases from a point of a front end to a portion connected to the main body.
